(19) United States
(12) Patent Application Publication
(10) Pub. No.: US 2002/0048585 A1
(43) Pub. Date: Apr. 25, 2002

(54) Methods of modulating wound healing and angiogenesis

(76) Inventor: Paul F. Goetinck, Boston, MA (US)

Correspondence Address: DIANA M. COLLAZO, Fish & Richardson P.C., 225 Franklin Street, Boston, MA, 02110-2804, US

(21) Appl. No.: 09/900,288

(22) Filed: Jul. 6, 2001

Related U.S. Application Data
(60) Provisional application No. 60/216,247, filed on Jul. 6, 2000.

(51) Int. Cl. A61K 48/00; A61K 39/395; A61K 38/17

(52) U.S. Cl. 424/155.1; 514/12; 514/44

(57) ABSTRACT

The invention features method of inhibiting angiogenesis in a subject. The method includes decreasing syndecan-4 activity or expression in a cell, tissue, or subject.

### RELATED APPLICATIONS

This application claims the benefit of U.S. Provisional Application Serial No. 60/216,247, filed on Jul. 6, 2000, which is incorporated herein by reference in its entirety.

### BACKGROUND

The syndecans make up a family of transmembrane heparan sulfate proteoglycans that act as co-receptors with integrins and growth factor tyrosine kinase receptors. Syndecan-4 is upregulated in skin dermis after wounding, and, in cultured fibroblasts adherent to the ECM protein fibronectin, this proteoglycan signals cooperatively with β1 integrins.

After skin injury, a dynamic process of tissue repair commences that consists of an inflammatory response followed by re-epithelialization of the wound area and establishment of the granulation tissue with accompanying neovascularization and wound contraction. The entire repair process is coordinated by highly regulated interactions of cells with their surrounding ECM and from their response to growth factors. Alterations in the composition of the ECM or growth factors can affect the wound healing process. In vitro, cells interact with the ECM molecule fibronectin through two types of cell-surface receptors: β1 integrins and the transmembrane heparan sulfate proteoglycan (HSPG) syndecan-4 (Woods et al. 1994. Mol. Biol. Cell. 5:183-192; Baciu et al. 1995. Mol. Biol. Cell. 6:1503-1513). Although cell adhesion to fibronectin is primarily dependent on β1 integrins that interact with the cell-binding domain of this ECM molecule, cooperative signaling from syndecan-4, as a result of an interaction with the heparin-binding domain of fibronectin, leads to the assembly of focal adhesions and actin stress fibers.

Syndecan-4 is detectable in the epidermis, but not in the dermis, of uninjured adult mouse skin. After skin injury, however, syndecan-4 is upregulated throughout the granulation tissue on endothelial cells and fibroblasts (Gallo et al. 1996. J. Invest. Dermatol. 107:676-683). Keratinocytes that migrate across the fibrin clot have reduced levels of syndecan-4 compared with the hyperproliferative keratinocytes distal from the wound edge.

### SUMMARY

The present invention is based, in part, on the inventor's work with mice in which the syndecan-4 gene is disrupted by homologous recombination in embryonic stem (ES) cells. Mice homozygous or heterozygous for the disrupted syndecan-4 gene have statistically significant delayed healing of skin wounds and impaired angiogenesis in the granulation tissue compared with wild-type littermates. These results indicate that modulation of syndecan-4 can affect wound healing and angiogenesis and that syndecan-4 is haplo-insufficient in these processes.

Accordingly, the invention features a method of modulating, e.g., promoting, angiogenesis in a subject, e.g., a human or a non-human animal. The method includes: increasing syndecan-4 activity, level, or expression in the subject.

In a preferred embodiment, syndecan-4 activity, level, or expression is increased by administering an agent which increases syndecan-4 expression.

In a preferred embodiment, the agent is a syndecan-4 polypeptide, or a biologically active fragment or analog thereof.

In a preferred embodiment, the agent is a nucleic acid encoding a syndecan-4 polypeptide, or a biologically active fragment or analog thereof.

In a preferred embodiment, the agent is an agonist of syndecan-4.

In a preferred embodiment, the agent is a small molecule which binds to the promoter region of syndecan-4.

In a preferred embodiment, syndecan-4 expression is increased by increasing endogenous syndecan-4 expression, e.g., by increasing transcription of the syndecan-4 gene.

In a preferred embodiment, transcription of the syndecan-4 gene is increased by altering the regulatory sequences of the endogenous syndecan-4 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the syndecan-4 gene to be transcribed more efficiently.

In a preferred embodiment, a cell that has been genetically modified to express a syndecan-4 polypeptide, or a fragment or an analog thereof is introduced into the subject.

In a preferred embodiment, the cell is selected from the group consisting of a fibroblast, a keratinocyte, an epithelial cell, and an endothelial cell.

In a preferred embodiment, the cell is: an autologous, allogeneic, or xenogeneic cell.

In a preferred embodiment the subject has a disorder or condition characterized by insufficient angiogenesis or which can be treated by increasing angiogenesis. Examples of such disorders include: vascular disorders; insufficient vascularization of tissue, e.g., heart tissue, e.g., in the case of heart attack, brain or central nervous tissue in the case of some strokes; wounds, e.g., skin wounds.

In a preferred embodiment the method can further include administering the ligand of a syndecan-4 co-receptor, e.g., administering a growth factor, which is a ligand of a growth factor receptor, e.g, FGF-2.

In another aspect, the invention features a method of modulating, e.g., inhibiting, angiogenesis in a subject. The method includes decreasing syndecan-4 expression, level, or activity, in a cell, e.g., a fibroblast or an endothelial cell, or a tissue of the subject.

In a preferred embodiment, syndecan-4 expression, level, or activity, is decreased by administering an agent which decreases syndecan-4 expression.

In a preferred embodiment, the agent which decreases syndecan-4 expression, level, or activity, is a peptide that binds to syndecan-4 (either intracellularly or extracellularly) and inhibits it.

In a preferred embodiment, the peptide is a soluble inhibitory form of a naturally occurring ligand.

In a preferred embodiment, the peptide is a soluble inhibitory form of fibronectin or a fragment thereof.

In a preferred embodiment, expression, level, or activity is decreased in a fibroblast or an endothelial cell.

In a preferred embodiment, the subject has a disorder or condition characterized by unwanted angiogenesis or which can be treated by decreasing angiogenesis.

In a preferred embodiment, the subject has a disorder characterized by unwanted cell proliferation.

In a preferred embodiment, the subject has cancer.

In a preferred embodiment, the cancer is characterized by the presence of a solid tumor.

In a preferred embodiment, the method also includes inhibiting a syndecan-4 co-receptor, e.g., FGF-2 receptor.

In a preferred embodiment, the co-receptor is FGF-2 receptor.

In a preferred embodiment, an anti-FGF-2 receptor antibody, anti-FGF-2 antibody, soluble ligand binding fragments of the FGF-2 receptor, or soluble co-receptor binding fragments of FGF-2 is administered to the subject.

In a preferred embodiment, the method further includes evaluating a control cell, tissue or subject that is not treated with the candidate compound.

In a preferred embodiment, the agent which decreases syndecan-4 expression, level, or activity, is selected from the group consisting of: a syndecan-4 nucleic acid molecule that can bind to cellular syndecan-4 mRNA and inhibit expression of the protein, e.g., a syndecan-4 antisense molecule or ribozyme, an antibody that specifically binds to syndecan-4 protein, e.g., an antibody which binds to a portion of the extracellular domain of syndecan-4 or an intrabody, and an agent which decreases syndecan-4 nucleic acid expression, e.g., a small molecule which binds the promoter of syndecan-4. Also included are peptides, e.g., other than antibodies, which bind to syndecan-4 (either intracellularly or extracellularly) and inhibit it, e.g., by preventing its binding to a natural ligand or by interfering with signal transduction. Examples of such molecules include soluble inhibitory forms, e.g., fragments or mutants, of naturally occurring ligands, e.g., fibronectin. Other examples include naturally or non-naturally occurring peptides selected by binding to syndecan-4.

In a preferred embodiment, the level of syndecan-4 can be decreased by decreasing the endogenous syndecan-4 expression, e.g., by decreasing transcription of the syndecan-4 gene.

In a preferred embodiment, transcription of the syndecan-4 gene can be decreased by: altering the regulatory sequences of the endogenous syndecan-4 gene, e.g., by the addition of a negative regulatory sequence (such as a DNA-binding site for a transcriptional repressor).

In a preferred embodiment, the cell is a fibroblast or an endothelial cell.

In a preferred embodiment the subject has a disorder or condition characterized by unwanted angiogenesis or which can be treated by decreasing angiogenesis. Examples of such disorders include: vascular disorders; psoriasis; or disorders characterized by unwanted cell proliferation, e.g., tumors, e.g., solid cancers, e.g., breast cancer, rhabdomyosarcoma, retinoblastoma, Ewing sarcoma, neuroblastoma, or osteosarcoma; blood-born tumors such as leukemia; benign tumors such as acoustic neuroma, hemangioma, neurofibroma, trachoma or pyogenic granuloma. In other preferred embodiments, the subject has an ocular disorder, e.g., ocular neovascular disease, diabetic retinopathy, corneal graft rejection, neovascular glaucoma and retrolental fibroplasia, or epidemic keratoconjunctivitis; rheumatoid arthritis; osteoarthritis; atherosclerosis; a hereditary disease such as Osler-Weber-Rendu disease, or hereditary hemorrhagic telangiectasia; chronic inflammation, e.g., from ulcerative colitis or Crohn's disease.

In a preferred embodiment the method can further include inhibiting a syndecan-4 co-receptor, e.g., a growth factor receptor, e.g., FGF-2 receptor. This can be accomplished by reducing the expression, level, or activity of a co-receptor, e.g., a growth factor receptor, e.g, FGF-2 receptor; by reducing the expression, level, or activity of a co-receptor's ligand, e.g., a growth factor, e.g, FGF-2; or by inhibiting the interaction of the co-receptor and its ligand, e.g., a growth factor receptor and its ligand, e.g., FGF-2 receptor and FGF-2. Reduction of the expression, level or activity can be accomplished by methods analogous to those discussed herein for reducing the expression, level or activity of syndecan-4. The interaction of the co-receptor and its ligand can be effected with agents which bind either the co-receptor or its ligand, e.g., antibodies to the co-receptor or ligand (e.g., anti-FGF-2 receptor antibodies or anti-FGF-2 antibodies), soluble ligand binding fragments of the co-receptor, or soluble co-receptor binding fragments or mutants of the ligand, or mutants of the ligand which bind but do not enable signal transduction. Peptides, e.g., other than antibodies, which bind to the co-receptor (either intracellularly or extracellularly) or its ligand and antagonize the interaction, e.g., by preventing binding or by interfering with signal transduction, can also be used. Examples of such molecules include soluble inhibitory forms, e.g., fragments or analogs, of naturally occurring ligands, e.g., FGF-2. Other examples include naturally or non-naturally occurring peptides selected by binding to the co-receptor.

In another aspect, the invention features a method of treating a subject, e.g., a human or a non-human animal, having a disorder characterized by unwanted angiogenesis, e.g., cancer. The method includes: determining if a subject is at risk for a disorder characterized by unwanted angiogenesis; and decreasing syndecan-4 expression, level, or activity, in a cell, e.g., a fibroblast or an endothelial cell, or a tissue of the subject.

In a preferred embodiment, syndecan-4 expression, level, or activity is decreased by administering an agent which decreases syndecan-4 expression.

In a preferred embodiment, the agent which decreases syndecan-4 expression, level, or activity is selected from the group consisting of: a syndecan-4 nucleic acid molecule that can bind to cellular syndecan-4 mRNA and inhibit expression of the protein, e.g., a syndecan-4 antisense molecule or ribozyme, an antibody that specifically binds to syndecan-4 protein, e.g., an antibody which binds to a portion of the extracellular domain of syndecan-4 or an intrabody, and an agent which decreases syndecan-4 nucleic acid expression, e.g., a small molecule which binds the promoter of syndecan-4. Also included are peptides, e.g., other than antibodies, which bind to syndecan-4 (either intracellularly or extracellularly) and inhibit it, e.g., by preventing its binding to a natural ligand or by interfering with signal transduction. Examples of such molecules include soluble inhibitory forms, e.g., fragments or analogs, of naturally occurring ligands, e.g., fibronectin. Other examples include naturally or non-naturally occurring peptides selected by binding to syndecan-4.

In a preferred embodiment, the level of syndecan-4 can be decreased by decreasing the endogenous syndecan-4 expression, e.g., by decreasing transcription of the syndecan-4 gene.

In a preferred embodiment, transcription of the syndecan-4 gene can be decreased by: altering the regulatory sequences of the endogenous syndecan-4 gene, e.g., by the addition of a negative regulatory sequence (such as a DNA-binding site for a transcriptional repressor).

In a preferred embodiment, the cell is an endothelial cell or a fibroblast.

In a preferred embodiment the method can further include inhibiting a syndecan-4 co-receptor, e.g., a growth factor receptor, e.g, FGF-2 receptor. This can be accomplished by reducing the expression, level, or activity of a co-receptor, e.g., a growth factor receptor, e.g, FGF-2 receptor; by reducing the expression, level, or activity of a co-receptor's ligand, e.g., a growth factor, e.g, FGF-2; or by inhibiting the interaction of the co-receptor and its ligand, e.g., a growth factor receptor and its ligand, e.g., FGF-2 receptor and FGF-2. Reduction of the expression, level or activity can be accomplished by methods analogous to those discussed herein for reducing the expression, level or activity of syndecan-4. The interaction of the co-receptor and its ligand can be effected with agents which bind either the co-receptor or its ligand, e.g., antibodies to the co-receptor or ligand (e.g., anti-FGF-2 receptor antibodies or anti-FGF-2 antibodies), soluble ligand binding fragments of the co-receptor, or soluble co-receptor binding fragments or mutants of the ligand, or mutants of the ligand which bind but do not enable signal transduction. Peptides, e.g., other than antibodies, which bind to the co-receptor (either intracellularly or extracellularly) or its ligand and antagonize the interaction, e.g., by preventing binding or by interfering with signal transduction, can also be used. Examples of such molecules include soluble inhibitory forms, e.g., fragments or analogs, of naturally occurring ligands, e.g., FGF-2. Other examples include naturally or non-naturally occurring peptides selected by be binding to the co-receptor.

In a preferred embodiment the subject has a disorder or condition characterized by unwanted angiogenesis or which can be treated by decreasing angiogenesis. Examples of such disorders include: vascular disorders; psoriasis; or disorders characterized by unwanted cell proliferation, e.g., tumors, e.g., solid cancers, e.g., breast cancer, rhabdomyosarcoma, retinoblastoma, Ewing sarcoma, neuroblastoma, or osteosarcoma; blood-born tumors such as leukemia; benign tumors such as acoustic neuroma, hemangioma, neurofibroma, trachoma or pyogenic granuloma. In other preferred embodiments, the subject has an ocular disorder, e.g., ocular neovascular disease, diabetic retinopathy, corneal graft rejection, neovascular glaucoma and retrolental fibroplasia, or epidemic keratoconjunctivitis; rheumatoid arthritis; osteoarthritis; atherosclerosis; a hereditary disease such as Osler-Weber-Rendu disease, or hereditary hemorrhagic telangiectasia; chronic inflammation, e.g., from ulcerative colitis or Crohn's disease.

In another aspect, the invention features a method of treating a subject having a disorder characterized by unwanted cell proliferation, or unwanted cell migration, e.g., of fibroblasts or endothelial cells. The method includes: determining if a subject is at risk for a disorder characterized by unwanted cell proliferation, e.g., cancer, or unwanted cell migration; and decreasing syndecan-4 activity, level, or expression in the subject.

In a preferred embodiment, syndecan-4 activity, level, or expression is decreased by administering an agent which decreases syndecan-4 expression or activity.

In a preferred embodiment, the agent which decreases syndecan-4 activity, level, or expression is selected from the group consisting of: a syndecan-4 nucleic acid molecule that can bind to cellular syndecan-4 mRNA and inhibit expression of the protein, e.g., a syndecan-4 antisense molecule or ribozyme, an antibody that specifically binds to syndecan-4 protein, e.g., an antibody which binds to a portion of the extracellular domain of syndecan-4 or an intrabody, and an agent which decreases syndecan-4 nucleic acid expression, e.g., a small molecule which binds the promoter of syndecan-4. Also included are peptides, e.g., other than antibodies, which bind to syndecan-4 (either intracellularly or extracellularly) and inhibit it, e.g., by preventing its binding to a natural ligand or by interfering with signal transduction. Examples of such molecules include soluble inhibitory forms, e.g., fragments, analogs, or mutants, of naturally occurring ligands, e.g., fibronectin. Other examples include naturally or non-naturally occurring peptides selected by binding to syndecan-4.

In a preferred embodiment, the level of syndecan-4 can be decreased by decreasing the endogenous syndecan-4 expression, e.g., by decreasing transcription of the syndecan-4 gene.

In a preferred embodiment, transcription of the syndecan-4 gene can be decreased by: altering the regulatory sequences of the endogenous syndecan-4 gene, e.g., by the addition of a negative regulatory sequence (such as a DNA-binding site for a transcriptional repressor).

In a preferred embodiment, the level of syndecan-4 can be decreased by decreasing the endogenous syndecan-4 expression.

In a preferred embodiment, the disorder is a tumor, e.g., a solid tumor, or benign tumor such as acoustic neuroma, neurofibroma, trachoma and pyogenic granuloma.

In a preferred embodiment, the disorder is a leukemia.

In a preferred embodiment the subject has a tumor, e.g., a solid cancer, e.g., breast cancer, rhabdomyosarcomas, retinoblastoma, Ewing sarcoma, neuroblastoma, or osteosarcoma; a blood-born tumor such as leukemia; a benign tumor such as acoustic neuroma, hemangioma, neurofibroma, trachoma or pyogenic granulomas. In other embodiments, the subject has an ocular disorde, e.g., ocular neovascular disease, diabetic retinopathy, corneal graft rejection, neovascular glaucoma and retrolental fibroplasia, or epidemic keratoconjunctivitis; rheumatoid arthritis; osteoarthritis; atherosclerosis; a hereditary disease such as Osler-Weber-Rendu disease, or hereditary hemorrhagic telangiectasia; chronic inflammation, e.g., from ulcerative colitis or Crohn's disease.

In a preferred embodiment the method can further include inhibiting a syndecan-4 co-receptor, e.g., a growth factor receptor, e.g, FGF-2 receptor. This can be accomplished by reducing the expression, level, or activity of a co-receptor, e.g., a growth factor receptor, e.g, FGF-2 receptor; by reducing the expression, level, or activity of a co-receptor's ligand, e.g., a growth factor, e.g, FGF-2; or by inhibiting the interaction of the co-receptor and its ligand, e.g., a gowth factor receptor and its ligand, e.g., FGF-2 receptor and FGF-2. Reduction of the expression, level.or activity can be accomplished by methods analogous to those discussed herein for reducing the expression, level or activity of syndecan-4. The interaction of the co-receptor and its ligand can be effected with agents which bind either the co-receptor or its ligand, e.g., antibodies to the co-receptor or ligand (e.g., anti-FGF-2 receptor antibodies or anti-FGF-2 antibodies), soluble ligand binding fragments of the co-receptor, or soluble co-receptor binding fragments or mutants of the ligand, or mutants of the ligand which bind but do not enable signal transduction. Peptides, e.g., other than antibodies, which bind to the co-receptor (either intracellularly or extracellularly) or its ligand and antagonize the interaction, e.g., by preventing binding or by interfering with signal transduction, can also be used. Examples of such molecules include soluble inhibitory forms, e.g., fragments, analogs, or mutants, of naturally occurring ligands, e.g., FGF-2. Other examples include naturally or non-naturally occurring peptides selected by be binding to the co-receptor.

In another aspect, the invention features a method of promoting wound healing in a subject, e.g., a human or a non-human animal. The method includes increasing syndecan-4 expression, level, or activity in a cell or tissue of the subject.

In a preferred embodiment, syndecan-4 expression, level, or activity is increased by administering an agent which increases syndecan-4 expression.

In a preferred embodiment, the agent is a syndecan-4 polypeptide, or a biologically active fragment or analog thereof.

In a preferred embodiment, the syndecan-4 polypeptide, or fragment or analog thereof is topically administered, e.g., in a cream, lotion, gel, or other topical carrier. In some embodiments, the syndecan-4 polypeptide, or fragment or analog thereof is administered on a bandage.

In a preferred embodiment, the agent is a nucleic acid encoding a syndecan-4 polypeptide, or a biologically active fragment or analog thereof.

In a preferred embodiment, the agent is an agonist of syndecan-4.

In a preferred embodiment, the agent is a small molecule which regulates transcription of syndecan-4, e.g., an agent that binds to the promoter region of syndecan-4.

In a preferred embodiment, syndecan-4 expression is increased by increasing endogenous syndecan-4 expression, e.g., by increasing transcription of the syndecan-4 gene.

In a preferred embodiment, the cell is an endothelial cell or a fibroblast.

In a preferred embodiment, transcription of the syndecan-4 gene is increased by altering the regulatory sequences of the endogenous syndecan-4 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the syndecan-4 gene to be transcribed more efficiently.

In a preferred embodiment, a cell that has been genetically modified to express a syndecan-4 polypeptide, or a fragment or an analog thereof is introduced into the subject.

In a preferred embodiment, the cell is selected from the group consisting of a fibroblast, a keratinocyte, an epithelial cell, and an endothelial cell.

In a preferred embodiment, the cell is: an autologous, allogeneic, or xenogeneic cell.

In a preferred embodiment the subject has a wound, e.g., a skin wound; an ocular disorder, e.g., ocular neovascular disease, diabetic retinopathy, corneal graft rejection, neovascular glaucoma and retrolental fibroplasia, or epidemic keratoconjunctivitis; rheumatoid arthritis; osteoarthritis; atherosclerosis; a hereditary disease such as Osler-Weber-Rendu disease, or hereditary hemorrhagic telangiectasia; chronic inflammation, e.g., from ulcerative colitis or Crohn's disease.

In a preferred embodiment the method can further include administering the ligand of a syndecan-4 co-receptor, e.g., administering a growth factor, which is a ligand of a growth factor receptor, e.g, FGF-2.

In another aspect, the invention features a method of evaluating if a subject is at risk for delayed wound healing. The method includes evaluating the presence of a syndecan-4 nucleic acid or protein. A decrease in syndecan-4 level, activity, or expression is indicative of the subject being at risk.

In a preferred embodiment, the presence of syndecan-4 is evaluated by contacting a biological sample, e.g., a skin sample, with a compound or an agent capable of detecting syndecan-4 protein or a syndecan-4 nucleic acid, such that the presence of syndecan-4 nucleic acid or protein is detected in the biological sample.

In a preferred embodiment, the compound or agent is a nucleic acid probe capable of hybridizing to syndecan-4 mRNA or an antibody capable of binding to syndecan-4 protein.

In another aspect, the invention features a method of identifying a compound which can be used to promote angiogenesis. The method includes: providing a cell, a tissue, or a subject; treating the cell or the tissue, or the subject with a candidate compound; and determining syndecan-4 level, activity, or expression, e.g., the level of syndecan-4 nucleic acid or syndecan-4 protein. The ability of the compound to increase syndecan-4 nucleic acid or syndecan-4 protein is indicative of a compound which can be used to promote angiogenesis.

In a preferred embodiment, the method also includes evaluating a control cell, tissue or subject that is not treated with the candidate compound.

In another aspect, the invention features a method of identifying a compound which can be used to promote wound healing. The method includes: providing a cell, a tissue, or a subject; treating the cell or the tissue, or the subject with a candidate compound; and determining syndecan-4 level, activity, or expression, e.g., the level of syndecan-4 nucleic acid or syndecan-4 protein. The ability of the compound to increase syndecan-4 nucleic acid or syndecan-4 protein is indicative of a compound which can be used to promote wound healing.

In a preferred embodiment, the method also includes evaluating a control cell, tissue or subject is not treated with the candidate compound.

In another aspect, the invention features a method of identifying a compound which can be used to inhibit angiogenesis. The method includes: providing a cell, a tissue, or a subject; treating the cell or the tissue, or the subject with a candidate compound; and determining syndecan-4 level, activity, or expression, e.g., the level of syndecan-4 nucleic acid or syndecan-4 protein. The ability of the compound to decrease syndecan-4 nucleic acid or syndecan-4 protein is indicative of a compound which can be used to inhibit angiogenesis.

In a preferred embodiment, the method also includes evaluating a control cell, tissue or subject is not treated with the candidate compound.

In another aspect, the invention features a kit for promoting wound healing or angiogenesis. The kit includes an agent that increases syndecan-4 activity or expression, e.g., a syndecan-4 agonist, e.g., a small molecule syndecan-4 agonist, or a syndecan-4 polypeptide, or a biologically active fragment or analog thereof; and instructions for use to promote wound healing or angiogenesis. In some preferred embodiments, the kit includes a bandage having on it, or impregnated with, the agent, e.g., for topical use in wound healing or promotion of angiogenesis.

In another aspect, the invention features, a transgenic animal, e.g., a rodent, e.g., a rat or mouse, that has disrupted expression of syndecan-4, e.g., a syndecan-4 knockout. In one embodiment, the transgenic animal also has a second transgene, e.g., a knockout, for a second gene, e.g., another syndecan, e.g., syndecan-l, or a growth factor co-receptor, e.g., FGF-2 receptor. Each transgene can be heterozygous or homozygous, thus the animal can be a double heterozygote, e.g., +/31 , +/−, a double homozygote, e.g., −/−, −/−, or homozygous for one gene and heterozygous for the other, e.g., +/−, −/−. Preferably, the transgenic animal exhibits delayed wound healing or impaired angiogenesis. The animal can be used in any of the screens or methods described herein.

“Transgenic” in the present context denotes a non-human animal in which one or more cells carry a recombinant DNA molecule. The recombinant DNA molecule can be, e.g., a knock out construct designed to reduce expression of a gene, e.g., a syndecan-4 gene, in the animal, or a construct encoding a polypeptide and/or a regulatory sequence designed to modulate, e.g., increase, expression of an endogenous or heterologous gene product in the animal. Preferably, the recombinant DNA molecule is integrated within the animal's chromosomes. Preferred non-human transgenic animals are mammals, e.g., rodents, e.g., mice, rats, or guinea pigs.

### DESCRIPTION OF THE DRAWINGS

FIG. 1. Generation and characterization of syndecan-4 recombinant mice. (a) Structure of the murine syndecan-4 gene, the targeting vector, and the targeted allele. Exons are numbered 1 through 5. This organization is similar to that reported by Tsuzuki et al.1997. J. Biochem. 122:17-24). Sa, SacI; Sp, SpeI; Av, AvrII; EV, EcoRV; B, BamHI; Xh, XhoI; Sn, SnaBI. SA, splice acceptor; IRES, internal ribosomal entry site; LACZ, β-galactosidase; NEO, neomycin resistance. (b) Southern blot analysis of ES clones. (c) Southern blot analysis of mice derived from matings between syndecan-4+/+ mice. Wild-type (14.1 kb) and targeted alleles (12 kb) are detected with the 5′ probe. (d) RT-PCR of mRNA results in a syndecan-4-specific 524-bp band from syndecan-4+/+ and syndecan-4+/− cells but not from syndecan-4−/− cells. mRNA for both β-actin and GAPDH is present in extracts of cells of all three genotypes. (e) Flow cytometric analysis of cell-surface syndecan-4 on skin fibroblasts. Syndecan-4 (Synd-4) is detected on syndecan-4+/+ cells (upper left) but not on syndecan-4−/− cells (upper right). β1 Integrins (β1) are expressed on cells of both genotypes (lower panels). Secondary antibodies alone were used as control (Cont).

FIG. 2. Delayed wound healing in mice homozygous (−/−) or heterozygous (+/−) for the recombinant syndecan-4 allele compared with wild-type (+/+) littermates. A statistically significant delay is seen between days 3 and 6 after wounding. Data are expressed as means ±SEM (n=20). ^{A}P<0.05. ^{B}P<0.01.

FIG. 3. Quantitative analysis of angiogenesis. The numbers of vessels per unit area are the same for the three genotypes (left). Average vessel size (middle) and total vessel area (right) in the wounds of syndecan-4−/− or syndecan-4+/− mice are significantly different from those of syndecan-4+/+mice. Data are expressed as means ±SEM (n=8). AP<0.05.

### DETAILED DESCRIPTION

Unwanted angiogenesis occurs in many disease states, tumor metastasis and abnormal growth by endothelial cells. These disorders are classified as angiogenic dependent or angiogenic associated diseases. Thus, therapies directed at control of angiogenesis are useful in the treatment of these diseases. Angiogenesis associated diseases include tumors, e.g., cancer, (e.g., solid cancers, e.g., breast cancer), blood-born tumors such as leukemias, and benign tumors such as acoustic neuroma, hemangioma, neurofibroma, trachoma and pyogenic granulomas; ocular disorders, e.g., ocular neovascular disease, diabetic retinopathy, corneal graft rejection, neovascular glaucoma and retrolental fibroplasias, or epidemic keratoconjunctivitis; rheumatoid arthritis; osteoarthritis; atherosclerosis; hereditary diseases such as Osler-Weber-Rendu disease, or hereditary hemorrhagic telangiectasia. Chronic inflammation may also involve pathological angiogenesis. Such disease states include ulcerative colitis and Crohn's disease. Angiogenesis is also prominent in solid tumor formation and metastasis.

Heterozygous (−/+) and homozygous (−/−) mice lacking the syndecan-4 gene were constructed as illustrated in Example 2. These mice and cells from these mice show impaired angiogenesis and wound healing. Impaired wound healing in syndecan-4−/− and syndecan-4+/− mice.

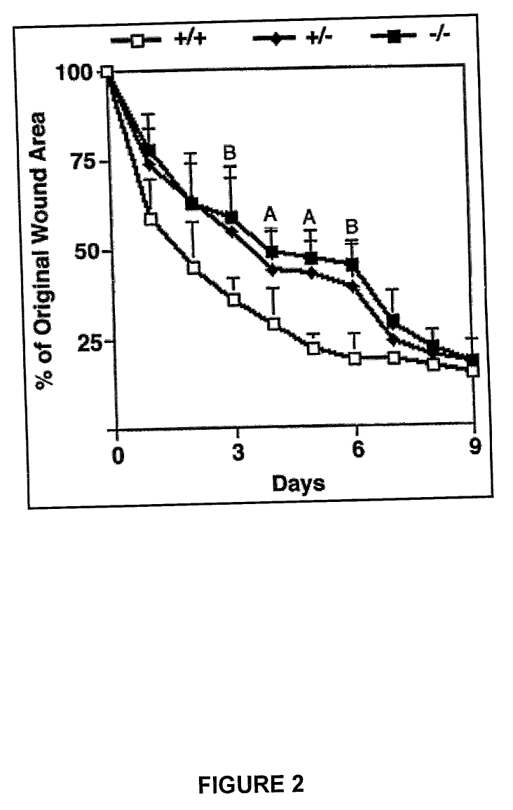

Excisional wounds were made on the backs of five 10-week-old female mice of each of the three genotypes. The wound areas of the of syndecan-4+/+ mice were reduced by more than 50% within 2 days and up to 75% within 4 days after wounding. In contrast, both syndecan-4−/− and syndecan-4+/− mice showed a delay in the rate of wound healing compared with syndecan-4+/+mice during the first 7 days after wounding (FIG. 2). These differences were statistically significant between days 3 and 6 after wounding. By day 9, all mice had closed their wounds to the same extent of about 80%. Complete wound closure in mice of all three genotypes was evident by day 12.

Day 3 after wounding was the first time point at which a statistically significant delay in the closure of the wounds of syndecan-4−/− and syndecan-4+/− mice was apparent. At this time point, comparable re-epithelialization had started at the edge of the wounds of the syndecan-4+/+and syndecan-4−/− mice as well as in the syndecan-4+/− mice. After 5 to 7 days, the new epidermis had formed completely and no differences were evident between the three genotypes. At 3 days, in the dermis of syndecan-4+/+ mice, there was an increase in the granulation tissue at the edge of the wound bed and the wounds of the syndecan-4+/+ mice had contracted. In contrast, the wounds of the syndecan-4−/− and syndecan-4+/− mice showed a greatly reduced accumulation of granulation tissue at the wound edge with a thin granulation tissue layer covering the wound bed. Unlike the wounds of the syndecan-4+/+ mice, those of the syndecan-4−/− and syndecan-4+/− mice did not contract at this time. By day 7, the wound bed was completely filled with granulation tissue in the dermis of all three genotypes, but the granulation tissue in the wounds of the syndecan-4+/+ mice seemed more vascularized than in those of the syndecan-4−/− and syndecan-4+/− mice.

Impaired angiogenesis in syndecan-4−/− and syndecan-4+/− mice

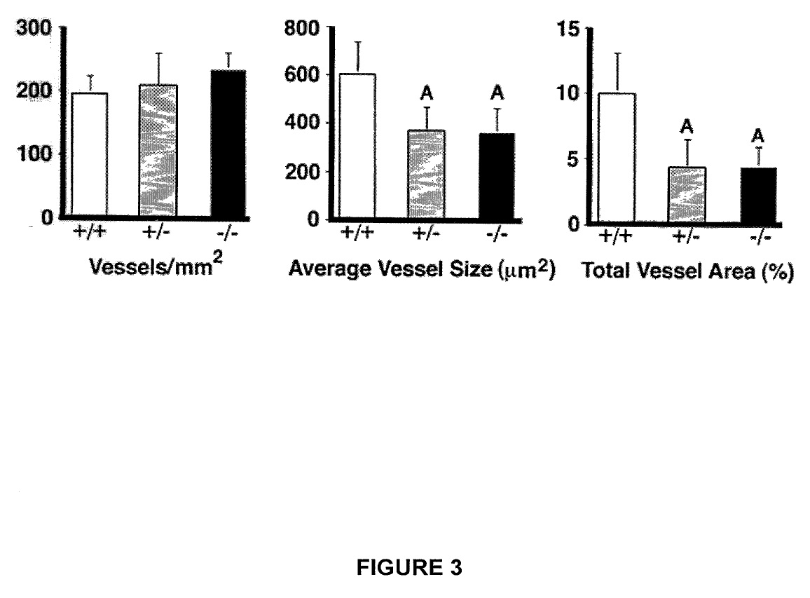

To quantify the differences in wound vascularization of the granulation tissues, eight female mice of each of the three genotypes were wounded and examined 6 days after wounding for the endothelial specific marker CD31. Immunohistological micrographs show greater staining for CD31 in the granulation tissue of syndecan-4+/+ mice compared with syndecan-4−/− and syndecan-4+/− mice. The number of vessels per unit area was comparable in the wounds of the three genotypes (FIG. 3, left). However, a statistically significant reduction (P<0.05) of the average vessel size in the granulation tissue of the syndecan-4−/− and syndecan-4+/− mice (FIG. 3c, middle) is evident. The smaller vessel size results in a statistically significant reduction in the relative wound area occupied by blood vessels, i.e., the total vessel area, for both syndecan-4−/− and syndecan-4+/− mice (FIG. 3c, right).

In vitro analyses of primary skin fibroblasts

Dermal skin fibroblasts, of all three genotypes, adherent to fibronectin spread and assemble an actin cytoskeleton and vinculin containing adhesion sites similar to those shown by Ishiguro et al. (2000. J. Biol. Chem. 275:5249-5252) for whole embryo derived fibroblasts.

In vitro wound migration assays reveal a slower rate of cell migration by the syndecan-4−/− fibroblasts compared with the syndecan-4+/+ or syndecan-4+/− fibroblasts cultured in DMEM containing 2% FBS. The syndecan-4+/+ or syndecan-4+/− fibroblasts had closed the wounds by 23 hours, whereas the syndecan-4−/− fibroblasts had not. No differences were observed between syndecan-4+/+ and syndecan-4+/− fibroblasts. Supplementing the medium with either 25 ng/ml of FGF-2 or with 25 ng/ml of EGF (FIG. 4, EGF) did not alter the rate of wound closure by cells of any of the three genotypes. In subcutaneous plug assays designed to test the migration of mouse endothelial cells into matrigel plugs supplemented with either VEGF165 or FGF-2, cells from the null mice migrate more slowly in the matrigel plugs than the wild type cells. Thus, the absence of cell surface syndecan-4 reduces the rate of migration of at least two different cell types, fibroblasts and endothelial cells.

Gel contraction assays reveal no differences for the three genotypes in their ability to contract collagen gels. The percent contraction of the gels (mean i SEM) at the end of 3 days was 75.12±10.96 (n=4), 79.21±7.37 (n=7), and 85.33±10.66 (n=3) for the syndecan-4+/+, syndecan-4+/−, and syndecan-4−/− fibroblasts, respectively. Student t tests indicate that these values are not statistically different from each other.

Because of their heparan sulfate side chains, heparan sulfate proteoglycans (HSPGs), e.g., syndecan-4, can bind a large number of bioactive molecules. In that capacity the HSPGs can act independently as a cell surface receptor or in a cooperative manner with other cell surface receptors. Syndecan-4 acts cooperatively with integrins in the assembly of focal adhesions and actin stress fibers when cells are plated on the ECM protein fibronectin and this cooperativity is Rho-mediated. In addition, syndecan-4 acts cooperatively with the receptor for FGF-2. Syndecan-4 null dermal fibroblasts proliferate more slowly than wild type cells and although they respond to FGF-2, activation of the MAPK pathway in the null fibroblasts is attenuated and of shorter duration compared to wild type cells. Furthermore, translocation of activated MAPK to the nucleus is not detectable in the mutant cells. We interpret these results to mean that the absence of syndecan-4 alters the signaling through the FGF-2 RTK.

Syndecan-4 is very widely expressed in the organism. In uninjured adult mouse skin syndecan-4 is expressed in the epidermis, but not in the dermis. After skin injury, however, syndecan-4 is up-regulated throughout the wound granulation tissue on both endothelial cells and fibroblasts. The hypothesis that syndecan-4 may play a role in wound repair was tested by generating mice in which the syndecan-4 gene was disrupted by homologous recombination in embryonic stem cells. It was found that mice homozygous or heterozygous for the disrupted syndecan-4 gene have statistically significant delayed healing of skin wounds as well as impaired angiogenesis in the granulation tissue compared to wild type littermates. The impaired angiogenesis is reflected in a statistically significant reduction in the size, but not the number, of the blood vessels in the granualtion tissue. In in vitro wound assays dermal fibroblast of the syndecan-4 null mice migration more slowly than wild type cells. Syndecan-4 null endothelial cells also migrate more slowly than wildtype cells. Thus, in subcutaneous plug assays designed to test the migration of mouse endothelial cells into matrigel plugs supplemented with either VEGF165 or FGF-2, we find that the cells from the null mice migrate more slowly in the matrigel plugs than the wild type cells. Thus, the absence of cell surface syndecan-4 reduces the rate of migration of at least two different cell types, fibroblasts and endothelial cells.

Examples that indicate syndecan-4 is involvement vascular conditions include elevation of plasma levels of syndecan-4 in individuals with acute myocardial infarction, upregulation of syndecan-4 after balloon angioplasty and upregulation of both VEGF and syndecan-4 in cardiac myocytes as a result of hypoxia.

Wound healing is a dynamic process in which the behavior of cells is influenced both by their interactions with the ECM and by their response to growth factors. Because syndecans function as co-receptors for both insoluble ligands such as components of the ECM and soluble ligands such as growth factors (Bernfield et al.1999. Annu. Rev. Biochem. 69:729-777), the observed delay in wound healing in syndecan-4−/− and syndecan-4+/− mice could result from an impairment of either or both of these functions. While not wanting to be bound by theory, possible explanations for the results described herein might be as follows. The reduced rate of migration of syndecan-4−/− fibroblasts in the in vitro wound healing model suggests that altered cell-matrix interactions could contribute to the delayed excisional wound healing. Syndecan-4 signals cooperatively with β1 integrins in the assembly of actin stress fibers and focal adhesions by ligating to the heparin-binding domain of fibronectin; yet, syndecan-4−/− fibroblasts adherent to fibronectin are indistinguishable from syndecan-4+/+ cells (Ishiguro et al.2000. J. Biol. Chem. 275:5249-5252). These results suggest that an unidentified receptor in syndecan-4−/− cells may interact with the heparin-binding domain of fibronectin and thus compensate for syndecan-4 deficiency in the assembly process, in agreement with Ishiguro et al. Such a compensatory signaling pathway might involve another member of the syndecan family and might explain why syndecan-4 null mice are indistinguishable from their wild-type littermates. Only under a stress situation, such as wound repair demonstrated in this study, would the role for syndecan-4 become evident.

The delayed wound repair in mice with the disrupted syndecan-4 gene could also result from alterations in the interactions of cells with growth factors such as FGF-2. FGF-2 null mice exhibit delayed wound healing (Ortega et al. 1998. Proc. Natl. Acad. Sci. USA. 95:5672-5677), and interactions of FGF-2 with syndecan-4 have been demonstrated (Volk et al. 1999. J. Biol. Chem. 274:24417-24424). Syndecan-4 null dermal fibroblasts described herein proliferate more slowly than wild type cells and although they respond to FGF-2, activation of the MAPK pathway in the null fibroblasts is attenuated and of shorter duration compared to wild type cells. Furthermore, translocation of activated MAPK to the nucleus is not detectable in the mutant cells, suggesting that the absence of syndecan-4 alters the signaling through the FGF-2 RTK.

Syndecan-1 is also upregulated during skin wound healing, but this upregulation is restricted to the endothelial cells of the granulation tissue (Gallo et al. 1996. J. Invest. Dermatol. 107:676-683). Lack of syndecan-1 results in delayed skin and corneal wound repair (Bernfield et al.1999. Annu. Rev. Biochem. 69:729-777; Stepp et al.1999. Mol. Biol. Cell. 10:459a). Like syndecan-4 null mice, syndecan-1 null mice are viable and fertile. An analysis of mice that are null for both syndecan-4 and -1 might shed light on whether these two HSPGs might provide complementary signaling pathways during wound healing.

The results presented herein clearly support the hypothesis that syndecan-4 plays a role in wound healing and angiogenesis and that syndecan-4 is haplo-insufficient for both of these processes. Therefore, modulation of syndecan-4 expression or activity can be useful in methods of modulating wound healing or angiogenesis, and/or in methods of treatment of wound-healing or angiogenesis-related disorders described herein. Syndecan-4 expression or activity can be modulated by, e.g., the use of anti-syndecan-4 antibodies; nucleic acid molecules, e.g., antisense molecules or nucleic acid molecules encoding synddecan-4; or agents, e.g., peptides, that act as agonists or antagonists of syndecan-4 activity or expression.

Syndecan expression or activity can be assayed, e.g., by assaying syndecan-4 mRNA or protein expression; assaying binding to the heparin-binding domain of ECM molecules, e.g., fibronectin; assaying the assembly of focal adhesions and actin stress fibers; assaying wound healing; assaying angiogenesis. These types of assays are routine in the art.

Antisense Nucleic Acid Molecules and Ribozymes

The methods described herein can comprise modulating, e.g., decreasing, syndecan-4 activity by antisense techniques. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire syndecan-4 coding strand, or to only a portion thereof (e.g., the coding region of a syndecan-4). In another embodiment, the antisense nucleic acid molecule is antisense to a “noncoding region” of the coding strand of a nucleotide sequence encoding a syndecan-4 (e.g., the 5′ and 3′ untranslated regions).

An antisense nucleic acid can be designed such that it is complementary to the entire coding region of syndecan-4 mRNA, but more preferably is an oligonucleotide that is antisense to only a portion of the coding or noncoding region of syndecan-4 mRNA. For example, the antisense oligonucleotide can be complementary to the region surrounding the translation start site of syndecan-4 mRNA, e.g., between the −10 and +10 regions of the target gene nucleotide sequence. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions with procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

The antisense nucleic acid molecules of the invention are typically administered to a subject (e.g., by direct injection at a tissue site), or generated in situ such that they hybridize with or bind to cellular mRNA and/or genomic DNA encoding a syndecan-4 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong polymerase II or polymerase III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a syndecan-4 -encoding nucleic acid can include one or more sequences complementary to the nucleotide sequence of a SYNDECAN-4 cDNA, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a syndecan-4 -encoding mRNA. See, e.g., Cech et al. U.S. Pat. No. 4,987,071; and Cech et al. U.S. Pat. No. 5,116,742. Alternatively, syndecan-4 mRNA can be used to select a catalytic RNA having a specific ribonuclease activity from a pool of RNA molecules. See, e.g., Bartel and Szostak (1993) Science 261:1411-1418.

Syndecan-4 gene expression can be inhibited by targeting nucleotide sequences complementary to the regulatory region of a syndecan-4 gene (e.g., the syndecan-4 promoter and/or enhancers) to form triple helical structures that prevent transcription of a syndecan-4 gene in target cells. See generally, Helene, C. (1991)Anticancer Drug Des. 6(6):569-84; Helene, C. et al. (1992) Ann. N.Y Acad. Sci. 660:27-36; and Maher, L. J. (1992) Bioassays 14(12):807-15. The potential sequences that can be targeted for triple helix formation can be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Transgenic Animals

The invention provides non-human transgenic animals. As used herein, a “transgenic animal” is a non-human animal, preferably a mammal, more preferably a rodent such as a guinea pig, rat, or mouse, in which one or more of the cells of the animal includes a transgene. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal. Other transgenes reduce or eliminate expression e.g., in a “knockout” animal. Preferred transgenic animals of the invention are rodents having reduced syndecan-4 expression, e.g., having a syndecan-4 transgene, e.g., as described herein. In some embodiments, the transgenic animals can have more than one transgene, e.g., the animals can have a syndecan-4 and an FGF transgene.

Methods for generating non-human transgenic animals, e.g., rodents, are known in the art. Such methods can involve introducing DNA constructs into the germ line of a non-human animal to make a transgenic mammal. For example, one or several copies of a knock out construct may be incorporated into the genome of a non-human embryo by standard transgenic techniques. Totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a preferred method, developing embryos are infected with a retrovirus containing the desired DNA, and transgenic animals produced from the infected embryo. In another preferred method, the appropriate DNAs are coinjected into the pronucleus or cytoplasm of embryos, preferably at the single cell stage, and the embryos allowed to develop into mature transgenic animals. Those techniques as well known. See reviews of standard laboratory procedures for microinjection of heterologous DNAs into mammalian fertilized ova, including Hogan et al., MANIPULATING THE MOUSE EMBRYO, (Cold Spring Harbor Press 1986); Krimpenfort et al., Bio/Technology 9:844 (1991); Palmiter et al., Cell, 41:343 (1985); Kraemer et al., GENETIC MANIPULATION OF THE EARLY MAMMALIAN EMBRYO, (Cold Spring Harbor Laboratory Press 1985); Hammer et al., Nature, 315:680 (1985); Wagner et al., U.S. Pat. No. 5,175,385; Krimpenfort et al., U.S. Pat. No. 5,175,384, the respective contents of which are incorporated by reference.

In addition, non-human transgenic mammals can be produced using a somatic cell as a donor cell. The genome of the somatic cell can then be inserted into an oocyte and the oocyte can be fused and activated to form a reconstructed embryo. For example, methods of producing transgenic animals using a somatic cell are described in PCT Publication WO 97/07669.

Antibodies

In another aspect, the invention features antibodies which inhibit syndecan-4 activity, to thereby treat a subject having a wound healing- or angiogenesis-related disorder.

An anti-syndecan-4 antibody or fragment thereof can be used to bind syndecan-4, e.g., the extracellular domain of syndecan-4, and thereby reduce syndecan-4 activity. Anti-syndecan-4 antibodies can be administered such that they interact with syndecan-4 protein locally at the site of alteration, e.g., at the cell memebrane, but do not inhibit syndecan-4 expression generally in the cell.

Syndecan-4 antibodies are known in the art and may be available commercially. Alternatively, the syndecan-4 protein, or a portion or fragment thereof, can be used as an immunogen to generate antibodies that bind syndecan-4 using standard techniques for polyclonal and monoclonal antibody preparation. The full-length syndecan-4 can be used or, alternatively, antigenic peptide fragments of syndecan-4 can be used as immunogens, e.g., a syndecan-4 extracellular domain can be used as an immunogen. In a preferred embodiment, the antibody binds to an extracellular domain of syndecan-4, or a portion thereof.

Typically, a syndecan-4 or a peptide thereof is used to prepare antibodies by immunizing a suitable subject, (e.g., rabbit, goat, mouse or other mammal) with the immunogen. An appropriate immunogenic preparation can contain, for example, syndecan-4 obtained by expression of the sequence encoding syndecan-4 or by gene activation, or a chemically synthesized syndecan-4 peptide. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881 which are hereby expressly incorporated by reference in their entirety. The preparation can further include an adjuvant, such as Freund's complete or incomplete adjuvant, or similar immunostimulatory agent. Immunization of a suitable subject with an immunogenic syndecan-4 preparation induces a polyclonal anti-target protein antibody response.

Anti-syndecan-4 antibodies or fragments thereof can be used as a syndecan-4 inactivating agent. Examples of anti- syndecan-4 antibody fragments include F(v), Fab, Fab′ and F(ab′)2 fragments which can be generated by treating the antibody with an enzyme such as pepsin. The term “monoclonal antibody” or “monoclonal antibody composition”, as used herein, refers to a population of antibody molecules that contain only one species of an antigen binding site capable of immunoreacting with a particular epitope of the target protein. A monoclonal antibody composition thus typically displays a single binding affinity for the particular target protein with which it immunoreacts.

Additionally, anti-syndecan-4 antibodies produced by genetic engineering methods, such as chimeric and humanized monoclonal antibodies, comprising both human and non-human portions, can be made using standard recombinant DNA techniques. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. No. 5,225,539; Jones et al., Nature 321:552-525, 1986; Verhoeyan et al., Science 239:1534, 1988; and Beidler et al., J. Immunol. 141:4053-4060, 1988.

In addition, a monoclonal antibody directed against syndecan-4 can be made using standard techniques. For example, monoclonal antibodies can be generated in transgenic mice or in immune deficient mice engrafted with antibody-producing cells, e.g., human cells. Methods of generating such mice are described, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with syndecan-4 or an antigenic syndecan-4 peptide and splenocytes from these immunized mice can then be used to create hybridomas. Methods of hybridoma production are well known.

Human monoclonal antibodies against syndecan-4 can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind the target protein, can be mutated, by for example using randomly altered mutagenized oligonucleotides, to generate a library of mutated variable regions which can then be screened to bind to the target protein. Methods of inducing random mutagenesis within the CDR regions of immunoglobulin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds syndecan-4. In a preferred embodiment, the primary screening of the library involves panning with the immobilized syndecan-4 and display packages expressing antibodies that bind the immobilized syndecan-4 are selected.

Display Libraries

The methods described herein can involve the use of peptides that modulate syndecan-4 activity, e.g., to thereby treat a subject having a wound-healing or angiogenesis related disorder, e.g., cancer. A display library can be screened to identify peptides that reduce syndecan-4 or peptides that increase, e.g., act as agonists of, syndecan-4 activity or expression.

In one approach for screening for syndecan-4 binding or modulating peptides, the candidate peptides are displayed on the surface of a cell or viral particle, and the ability of particular cells or viral particles to bind syndecan-4 via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands can be used to detect homologs that retain ligand-binding activity. The use of fluorescently labeled ligands allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and fl are most often used in phage display libraries. Either of the phage gIII or gVIII coat proteins can be used to generate fusion proteins without disrupting the ultimate packaging of the viral particle. Foreign epitopes can be expressed at the NH2-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Administration

The agents described herein for the promotion of wound healing or modulation of angiogenesis may be administered via the parenteral route, including orally, topically, subcutaneously, intraperitoneally, intramuscularly, intranasally, and intravenously. Topical administration is preferred for methods used to promote wound healing. Repeated administration of the agents, e.g., repeated topical administration, can be used. More than one route of administration can be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the syndecan-4 modulating agents.

Topical administration of the syndecan-4 modulating agents described herein presents a preferred route of administration amongst the many different routes described above for promotion of wound healing. For topical application, the methods and kits of the present invention can include the use of a medium compatible with skin. Such topical pharmaceutical compositions can exist in many forms, e.g., in the form of a solution, cream, ointment, gel, lotion, shampoo, or aerosol formulation adapted for application to the skin, e.g., to wounded skin. A wide variety of carrier materials can be employed for the syndecan-4 modulating agents described herein such as aloe vera gel, allantoin, glycerine, vitamin A and E oils, mineral oils, and polyethylene glycols. Other additives, e.g., antibiotics, can be included. The topical composition can be applied and removed immediately, or it can be applied and left on the skin surface, e.g., on a wound surface, for an extended period of time, e.g., overnight or throughout at least one day.

Administration of the agents discussed herein can be systemic or local. Local administration can be directed to the site of a disorder or lesion, e.g., to a site in need of vascularization, e.g., to heart or vascular tissue at or near the site of an obstruction, or to the site of a tumor, e.g. to the vasculature associated with a tumor.

Bandages and kits

In addition, the methods described herein can be used in non surgical wound healing applications, e.g., a bandage impregnated with an agent that modulates syndecan-4 expression or activity can be used for wound healing in addition to, or in place of, a conventional bandage. In one embodiment, a biocompatible substrate, e.g., a conventional bandage material, e.g., a strip of fiber, can be impregnated with a syndecan-4 modulating agent described herein, e.g., a peptide, and applied to a wound. The bandage can contain another beneficial material for wound healing, e.g., an antibiotic. In some embodiments, the bandage can be supplied to a subject in a kit, e.g., a kit for use by a health care practitioner, or a kit for household use, which kits can contain instructions for use. The bandage described herein can be left on the wound, or can be replaced as necessary.

Such a bandage can be used ex-vivo, on a tissue removed from the body, or in situ on a subject, e.g., a human subject. For example, a bandage described herein can be used as an “artificial skin” or covering agent to cover large, oozing surfaces inside or outside the body. Burn patients, for example, could be covered with a bandage described herein to assist in preventing bacterial infection and to lessen the loss of body fluids and electrolytes through the burned areas.

The invention also includes kits for use in wound healing or modulating angiogenesis. Such kits can be used for laboratory or for clinical applications. Such kits include an agent that modulates, e.g., increases, syndecan-4 expression or activity, e.g., a syndecan-4 peptide agonist as described herein, and instructions for applying the agent to repair or heal an animal tissue, e.g., a human tissue, particularly in a human patient. The kits can include a container for storage of the syndecan-4 modulating agent. Optionally, a kit can include an additional agent for use in a wound repair, e.g., an antibiotic.

The kits described herein can also include a means to apply the syndecan-4 modulating agent to a tissue, for example, a syringe or syringe-like device, a dropper, a powder, an aerosol container, and/or a bandage material.

Kits can include instructions for use, e.g., instructions for topical administration of the syndecan-4 modulating agent.

Incorporation by reference

All patents and references cited herein are hereby incorporated by reference in their entirety.

### EXAMPLES

### Example 1: Methods

Construction of the syndecan-4 targeting vector

A 120-kb genomic clone was isolated from a 129/SVJ BAC library by hybridization with the complete murine syndecan-4 cDNA. The IRESβgeo cassette was excised from the GT1.8 IRESβgeo plasmid (Mountford et al.1994. Proc. Natl. Acad. Sci. USA. 91:4303-4307) with SalI, the ends filled with Klenow polymerase, and cloned blunt end into the NruI digested PsLII80. A 4.5-kb genomic EcoRV/BamHI fragment containing exon 5 was cloned blunt end into the SnaBI site 3′ of the IRESβgeo cassette. A 6.4-kb SacI fragment from the first intron was blunt ended by T4 polymerase and cloned into the blunt ended XhoI site located 5′ of the IRESβgeo cassette. The orientation of all cloned fragments was verified by restriction digest analysis.

Embryonic stem cells and generation of homozygous mice

Twenty million 129/J1 ES cells were electroporated with 25 μg/ml of SpeI linearized targeting vector and subjected to G418 selection. G418-resistant clones were screened for homologous recombination of the syndecan-4 locus by Southern blot hybridization. Two recombinant clones were expanded, reanalyzed by Southern blot hybridization, and injected into C57BL/6 blastocysts. Chimeric male founder mice were backcrossed to C57BL/6 females, and heterozygous offsprings were backcrossed on that background for at least four generations. Genotype analysis was performed by Southern blot hybridization on genomic DNA isolated from tails.

Isolation of dermal fibroblasts

Primary skin fibroblasts were isolated from newborn syndecan-4+/+, syndecan-4+/−, and syndecan-4−/− mice from the same litter. Mice were sacrificed, and their skins were removed, flattened, and floated on a 0.25% trypsin solution (Life Technologies Inc., Gaithersburg, Md., USA) at 4° C. overnight. The dermis was separated from the epidermis and dissociated by stirring at room temperature for 30 minutes in HBSS containing collagenase (500 μg/ml) (Life Technologies Inc.). The cell suspension of each skin was passed through a cell strainer (Becton Dickinson and Co., Franklin Lakes, N.J., USA) and transferred to a 10-cm cell culture dish and cultured as described previously (Saoncella et al.1999. Proc. Natl. Acad. Sci. USA. 96:2805-2810). Dermal fibroblasts were maintained at subconfluent densities and used between passages three and six.

RT-PCR

RT-PCR was performed on 1 μg of total RNA from syndecan-4+/+, syndecan-4+/−, and syndecan-4−/− fibroblasts as described in Saoncella (supra). PCR reactions were performed with primers specific for syndecan-4 (forward: nucleotide [NT]+76 to NT+100 and reverse: NT+588 to +609), β-actin (forward: NT+73 to NT+85 and reverse: NT+993 to NT+1006), and GAPDH (CLONTECH Laboratories Inc., Palo Alto, Calif., USA). Bands were analyzed on 1.5% agarose gel, visualized by ultraviolet activation of incorporated ethidium bromide, and photographed.

Flow cytometry

Analysis of surface expression of syndecan-4 and the integrin β1 chain was performed as described in Saoncella (supra) using affinity-purified anti-mouse syndecan-4 antibodies (MS-4-E) and a rat mAb directed against the mouse β1 integrin chain (9EG7).

Wound healing studies

Four excisional full-thickness wounds were made with 6-mm skin biopsy punches on the backs of three groups of five 10-week-old female mice that were either syndecan-4+/+, syndecan-4+/−, or syndecan-4−/−. Wound closure was monitored daily as described in Streit et al. (2000. EMBO J. 19:3272-3282). Area measurements were made using the IP-LAB software (Scanalytics, Fairfax, Va., USA). Histological analysis of the wounds was performed on mice that were sacrificed at 3 and 7 days after wounding. In a separate experiment, wounds were created on eight 10-week-old female mice of each of the three genotypes, and the skins were collected at 6 days after wounding for the analysis of blood vessels. Statistical analysis was performed using the unpaired Student's t test.

Histology and Immunocytochemistry

Tissues were fixed in 4% paraformaldehyde for 2 hours and embedded in paraffin. Staining with hematoxylin and eosin was done on 6-μm paraffin sections. Blood vessels were analyzed on 6-μm cryostat sections stained with an mAb against mouse CD31 (PECAM-1) (PharMingen, San Diego, Calif., USA) as described in Detmar et al. (2000. Am. J. Pathol. 156:159-167). Image analysis was performed as described in Streit et al (supra) and the number of vessels, average vessel size, and total vessel area were determined. Statistical analysis was performed using the unpaired Student's t test. The assembly of focal adhesions and actin stress fibers was assessed by confocal microscopic analysis of vinculin and actin stress fibers in fibroblasts adherent to fibronectin for 3 hours in the presence of 10% FBS (10).

In vitro wound healing models

Primary fibroblast cultures were established for 2 days in DMEM with 10% FBS followed by an overnight culture in DMEM with 2% FBS. In vitro wound closure tests were performed by creating clear lines in confluent cultures with a sterile yellow plastic pipette tip. After two washes with medium, the monolayers were covered with fresh medium and the migration of the cells into the cleared spaces was monitored over time. The control medium used during wound closure was DMEM with 2% FBS. This medium was supplemented with either 25 ng/ml of FGF-2 (R&D Systems Inc., Minneapolis, Minneapolis, USA) or 25 ng/ml of EGF (Becton Dickinson and Co.).

In vitro wound contraction assays (Grinnell. 1994. J. Cell Biol. 124:401-404) were done by allowing 2 ml of DMEM with 10% FBS, 1.0 mg/ml of bovine type I collagen (Vitrogen 100; Collagen Corp., Palo Alto, Calif., USA) and 6.7×10⁴ fibroblasts to form a gel in 35-mm dishes for 3 hours at 37° C. before releasing the gels from the dish. Skin fibroblast populations were derived from four syndecan-4+/+, seven syndecan-4+/−, and three syndecan-4−/− newborn mice. Assays were performed in triplicate for each of the 14 different fibroblast populations. Gel contraction was quantified by measuring the diameter of the floating gels.

### Example 2. Establishment and characterization of syndecan-4-deficient mice.

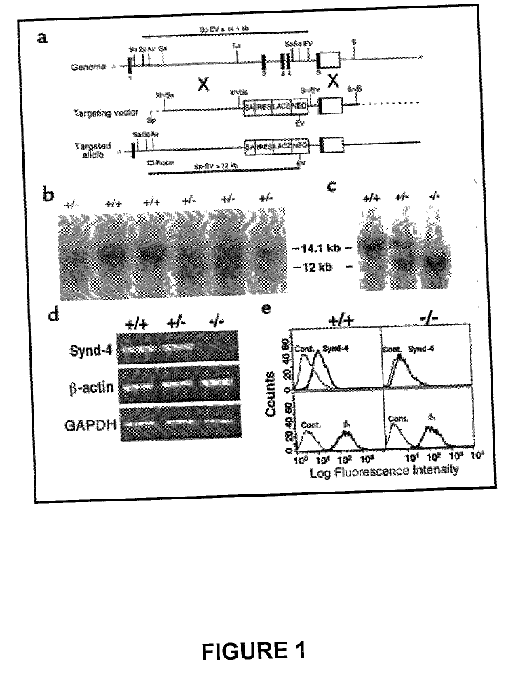

Of 200 clones selected as described in Example 1, 38 were screened by Southern blot hybridization of genomic DNA digested with SpeI and EcoRV using a 5′ external probe indicated in FIG. 1a. Of these, 17 clones were determined to carry the recombinant syndecan-4 allele (synd-4tm1 Goe) based on the presence of predicted 12-kb recombinant and 14-kb wildtype allele fragments (FIG. 1b). Two clones (13 and 27) were expanded, reanalyzed by Southern blot hybridization, and injected into C57BL/6 blastocysts. Chimeric male founder mice were backcrossed to C57BL/6 females, and the genotype of carriers of the recombinant allele were identified by Southern blot hybridization (FIG. 1c) and/or PCR analysis on genomic DNA isolated from tails. Mice homozygous and heterozygous for the disrupted syndecan-4 core protein gene develop normally, are fertile, and show no overt morphological defects. The mutant syndecan-4 gene segregated in a normal mendelian fashion, yielding approximately 25% homozygous mutant offspring in matings between mice heterozygous for the recombinant allele.

RT-PCR analysis of mRNA from skin fibroblasts obtained from 1-day-old sibling offspring of heterozygous parents revealed that a 524-bp syndecan-4-specific band could be amplified from syndecan-4+/+ and syndecan-4+/− cells but not from syndecan-4−/− cells. The predicted 946-bp β-actin and 983-bp GAPDH bands were amplified from mRNA isolated from cells of all three genotypes (FIG. 1d). A shift in fluorescence intensity for the syndecan-4+/+ cells (FIG. 1e, upper left) with the anti-syndecan-4 antibodies indicates the presence of syndecan-4 on these cells. The absence of a shift with the syndecan-4−/− cells (FIG. 1e, upper right) indicates an absence of syndecan-4 on these cells, confirming that the syndecan-4 gene had been disrupted. Both the syndecan-4+/+ (FIG. 1e, lower left) and syndecan-4−/− (FIG. 1e, lower right) cells show an identical shift in fluorescence intensity with the anti-β1 antibodies.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inhibiting angiogenesis in a subject comprising decreasing syndecan-4 expression, level, or activity, in the subject.

2. The method of claim 1, wherein syndecan-4 expression, level, or activity, is decreased by administering an agent which decreases syndecan-4 expression.

3. The method of claim 1, wherein the agent which decreases syndecan-4 expression, level, or activity, is selected from the group consisting of: a syndecan-4 nucleic acid molecule that can bind to cellular syndecan-4 mRNA and inhibit expression of the protein, an antibody that specifically binds to syndecan-4 protein, and an agent which decreases syndecan-4 nucleic acid expression.

4. The method of claim 1, wherein the agent which decreases syndecan-4 expression, level, or activity, is selected from the group consisting of: peptides that bind to syndecan-4 (either intracellularly or extracellularly) and inhibit it.

5. The method of claim 4 wherein the peptide is a soluble inhibitory form of a naturally occurring ligand.

6. The method of claim 4 wherein the peptide is a soluble inhibitory form of fibronectin or a fragment thereof.

7. In a preferred embodiment expression, level, or activity is decreased in a fibroblast or an endothelial cell.

8. The method of claim 1, wherein the subject has a disorder or condition characterized by unwanted angiogenesis or which can be treated by decreasing angiogenesis.

9. The method of claim 1, wherein the subject has a disorder characterized by unwanted cell proliferation.

10. The method of claim 1, wherein the subject has cancer.

11. The method of claim 10, wherein the cancer is characterized by the presence of a solid tumor.

12. The method of claim 1, further comprising inhibiting a syndecan-4 co-receptor.

13. The method of claim 12, wherein the co-receptor is FGF-2 receptor.

14. The method of claim 13, wherein an anti-FGF-2 receptor antibody, anti-FGF-2 antibody, soluble ligand binding fragment of the FGF-2 receptor, or soluble co-receptor binding fragment of FGF-2 is administered to the subject.

15. A method of identifying a compound which can be used to inhibit angiogenesis, comprising:
providing a cell, a tissue, or a subject;
treating the cell or the tissue, or the subject with a candidate compound; and
determining the level of syndecan-4 nucleic acid or syndecan-4 protein, wherein the ability of the compound to decrease sydecan-4 nucleic acid or syndecan-4 protein is indicative of a compound which can be used to inhibit angiogenesis.

16. The method of claim 15, further comprising evaluating a control cell, tissue or subject that is not treated with the candidate compound.
